(19) United States
(12) Patent Application Publication
(10) Pub. No.: US 2023/0129166 A1
(43) Pub. Date: Apr. 27, 2023

(54) SYSTEM AND METHOD FOR WEARABLE DEVICE CONTACT FORCE ESTIMATION AND ADJUSTMENT FEEDBACK

(71) Applicant: THE ROYAL INSTITUTION FOR ADVANCEMENT OF LEARNING/MCGILL UNIVERSITY, Montréal (CA)

(72) Inventors: Jeremy R. Cooperstock, Westmount (CA); Pascal E. Fortin, Montréal (CA); Jeffrey R. Blum, Montreal (CA); Antoine Weill-Duflos, Verdun (CA)

(21) Appl. No.: 17/973,369

(22) Filed: Oct. 25, 2022

Related U.S. Application Data
(60) Provisional application No. 63/271,396, filed on Oct. 25, 2021.

(51) Int. Cl. A61B 5/00; A61B 5/024

(52) U.S. Cl. CPC A61B 5/6802; A61B 5/02438; A61B 5/02427; A61B 2562/0247

(57) ABSTRACT

There is provided a contact force determination method comprising obtaining, at a computing device, at least one physiological signal indicative of at least one physiological parameter of a user, the at least one physiological signal obtained from at least one contact-based physiological sensor embedded in a wearable device secured to a body part of the user; determining, at the computing device, one or more statistical parameters of the at least one physiological signal; determining at the computing device, based on the one or more statistical parameters, a contact force between the wearable device and the body part; and outputting, at the computing device, the contact force as determined.

### CROSS-REFERENCE TO RELATED APPLICATIONS

This patent application claims priority of U.S. Provisional Application No. 63/271,396, filed on Oct. 25, 2021, the entire contents of which are hereby incorporated by reference.

### FIELD

This disclosure generally relates to the field of wearable devices, and more particularly to providing contact force estimation and adjustment feedback for wearable devices.

### INTRODUCTION OR BACKGROUND

Modern wearable devices are equipped with increasingly advanced physiological sensing hardware. Such wearable devices need to be firmly secured to a user's body for optimal performance, yet remain comfortable when worn for extended periods of time. In order to enhance device performance, users can manually adjust the tightness of their wearable devices. However, this manual and subjective tightness adjustment is unlikely to ensure repeatable data collection conditions and yield optimal results in all instances. While dedicated sensors may be used to directly measure or estimate the contact force between the wearable device and the user's body, use of such sensors results in an overall increase in complexity and cost. In addition, although automated software-based techniques may be used to detect and correct sensor signal anomalies and improve signal quality, these techniques sometimes fail to fully compensate for poor data quality.

As such, there is room for improvement.

### SUMMARY

In accordance with one aspect, there is provided a contact force determination method comprising obtaining, at a computing device, at least one physiological signal indicative of at least one physiological parameter of a user, the at least one physiological signal obtained from at least one contact-based physiological sensor embedded in a wearable device secured to a body part of the user; determining, at the computing device, one or more statistical parameters of the at least one physiological signal; determining at the computing device, based on the one or more statistical parameters, a contact force between the wearable device and the body part; and outputting, at the computing device, the contact force as determined.

In accordance with another aspect, there is provided a contact force determination system comprising a processing unit and a non-transitory computer-readable medium having stored thereon program instructions executable by the processing unit for obtaining at least one physiological signal indicative of at least one physiological parameter of a user, the at least one physiological signal obtained from at least one contact-based physiological sensor embedded in a wearable device secured to a body part of the user; determining one or more statistical parameters of the at least one physiological signal; determining, based on the one or more statistical parameters , a contact force between the wearable device and the body part; and outputting the contact force as determined.

In accordance with another aspect, there is provided a non-transitory computer-readable medium having stored thereon program instructions executable by a processor for obtaining at least one physiological signal indicative of at least one physiological parameter of a user, the at least one physiological signal obtained from at least one contact-based physiological sensor embedded in a wearable device secured to a body part of the user; determining one or more statistical parameters of the at least one physiological signal; determining, based on the one or more statistical parameters, a contact force between the wearable device and the body part; and outputting the contact force as determined.

Many further features and combinations thereof concerning embodiments described herein will appear to those skilled in the art following a reading of the instant disclosure.

### DESCRIPTION OF THE FIGURES

In the figures,

FIG. 1 is a schematic diagram illustrating a system for providing contact force estimation and adjustment feedback for a wearable device, in accordance with an illustrative embodiment;

FIGS. 2A and 2B are schematic diagrams of the wearable device of FIG. 1 in contact with a user's skin, in accordance with an illustrative embodiment;

FIG. 3 is a flowchart illustrating an example method for providing contact force estimation and adjustment feedback for a wearable device, in accordance with an illustrative embodiment; and

FIG. 4 is a block diagram of an example computing device, in accordance with an illustrative embodiment.

It will be noted that throughout the appended drawings, like features are identified by like reference numerals.

### DETAILED DESCRIPTION

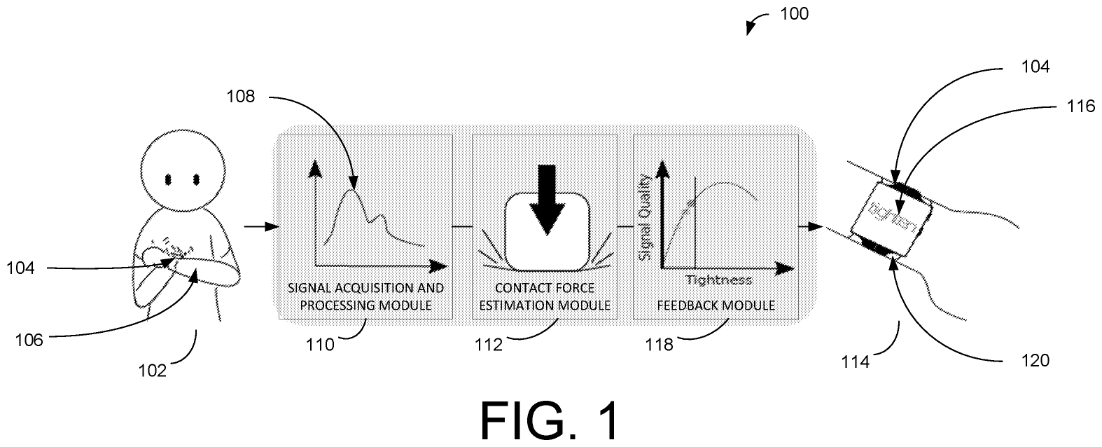

FIG. 1 illustrates a system 100 for providing contact force estimation and adjustment feedback for a wearable device. In FIG. 1, a user 102 wears a wearable device 104 in contact with his or her skin 106, the wearable device 104 secured to any suitable body part(s) of the user 102, for instance the user's wrist as illustrated. As used herein, the term “wearable device” refers to any suitable electronic device configured to be worn by a user as in 102. The wearable device 104 may include a variety of components. For example, the wearable device 104 may include one or more input devices (not shown) that the user 102 may manipulate by touch. The wearable device 104 may also include one or more output devices (including, but not limited to, a display and a speaker, not shown) for outputting information to the user 102. As will be discussed further below, the wearable electronic device 104 may include various sensors, such as sensors that may be used to detect information (e.g., physiological information) about the user 102. In addition, the wearable device 104 may include one or more bands, straps, or other suitable attachment mechanisms that may be used to attach the wearable device 104 to the user's body part(s) (e.g., the user's wrist).

The user 102 may manually adjust a tightness of the wearable device 104, in order to optimize the user's comfort level. For instance, the user 102 can adjust the tightness of a strap 120 securing the wearable device 104 to the skin 106, the wearable device 104 exerting a contact force (not shown) on the skin 106. As understood by those skilled in the art, a “contact force” is a force that is applied by objects in contact with each other, the contact force acting on a point of direct contact between the two objects. As used herein, the term “contact force” therefore refers to a normal force between the wearable device 104 and the body part the wearable device 104 is secured to. In other words, the contact force refers to the normal force exerted by the wearable device 104 on the body part and/or the normal force exerted by the body part on the wearable device 104). The contact force can be continuous (i.e. as a continuous force) or momentary (i.e. as an impulse). As used herein, the term “tightness” (also referred to as “tightness level”, “coupling rate” or “coupling force”) refers to the degree of coupling (or fitting) of the wearable device 104 to the user's body part. Such coupling is referred to herein as “tight” when the wearable device 104 is fitted to the body part in a manner that limits motion or movement of the wearable device 104 relative to the body part. The coupling is referred to herein as “loose” when the wearable device 104 is fitted to the body part in a manner that allows for some motion or movement of the wearable device 104. The tightness level is typically selected as a trade-off between adequately securing the wearable device 104 to the user's body part (to prevent undesirable motion and/or improper operation of the wearable device 104) and preventing discomfort or interference with the user's daily activities (e.g., due to an excessively tight wearable device 104).

Although the wearable device 104 is illustrated in FIG. 1 as a smartwatch secured to the user's wrist, it should be understood that other embodiments may apply. The wearable device 104 may indeed comprise any suitable wearable device, including, but not limited to, a fitness tracker, a smart medical device, a haptic system, a head-mounted display (HMD) for augmented reality (AR), virtual reality (VR) or extended reality (XR) applications, a video game controller, a smartphone, a smart garment, and the like. Furthermore, the wearable device 104 may be secured to (i.e. provided on or around) other parts of the user's body including, but not limited to, the user's finger(s), arm(s), ankle(s), leg(s), head, or face.

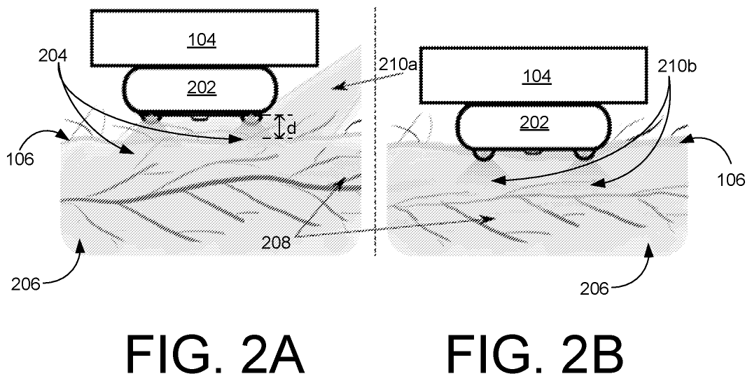

Referring now to FIGS. 2A and 2B in addition to FIG. 1, the wearable device 104 comprises at least one contact-based physiological sensor 202. In the illustrated embodiment, the physiological sensor 202 is embedded in the wearable device 104 and is configured to be positioned in contact with the skin 106 of the user 102. When so positioned, the physiological sensor 202 measures at least one physiological parameter of the user 102 and outputs at least one corresponding physiological sensor signal 108. In one embodiment, the signal 108 is indicative of measured values of the physiological parameter over time. According to one embodiment illustrated in FIG. 2A and FIG. 2B, the physiological sensor 202 is an optical heart rate monitor configured to measure the user's photoplethysmogram (PPG).

It should however be understood that contact-based physiological sensors other than optical heart rate (or PPG) sensors may also apply. In addition, in some embodiments, the wearable device 104 may comprise one or more additional sensors using any suitable sensing technology and configured to provide one or more additional sensor signals. The one or more additional sensor signals may then be used to perform contact estimation and adjustment feedback, in a similar manner to that described herein with reference to the physiological sensor 202. For example, sensing technologies including, but not limited to, skin conductance sensors (configured to measure conductance of the skin 106), motion sensors (configured to detect movement of the user's body part), accelerometers (configured to detect body part orientation and acceleration), gyroscopes (configured to measure orientation and angular velocity), altimeters (configured to measure the user's altitude above a fixed level such as ground), or the like, may apply.

The physiological sensor 202, exemplarily shown in FIGS. 2A and 2B as an optical heart rate monitor, may be configured to emit (e.g., using a light-emitting diode (LED), not shown) light 204 towards the skin 106 of the user 102. The light 204 is reflected and transmitted through the user's tissues 206 and blood vessels 208, and examined by the sensor 202 to determine the user's heart rate. Since blood absorbs light, fluctuations in light level can be translated into heart rate, a process referred to as photoplethysmography. As understood by those skilled in the art, the accuracy of optical heart rate sensors is sensitive to how tightly the sensors are pressed against a user's skin. The changes in reflection and transmission of the light 204 through the tissues 206 and blood vessels 208 may therefore be monitored to estimate contact force. The tissues 206 and blood vessels 208 may be in either an uncompressed state (as shown in FIG. 2A) or a compressed state (as shown in FIG. 2B), depending on the level of tightness of the wearable device 104 against the user's skin 106. FIGS. 2A and 2B illustrate the effects of the tightness of the wearable device 104 on the quality of the signal acquired by the physiological sensor 202. If the wearable device 104 is not worn adequately tightly, the device's performance may deteriorate to a point where the device 104 may become unusable.

In the embodiment of FIG. 2A, the wearable device 104 is insufficiently tightened on the user's skin 106 (e.g., with a spacing d being created between the skin 106 and the sensor 202), resulting in the physiological sensor 202 being loosely coupled to the user's skin 106 and in the tissues 206 and blood vessels 208 being left in an uncompressed state. Loose coupling may result in the pulsatile component of the physiological sensor signal 108 becoming negligible in comparison with the attenuation caused by the thickness of the soft tissues as in 206. Loose coupling may also let user's motion disturb the position of the physiological sensor 202, introducing artifacts into the data acquired by the physiological sensor 202. Parasitic light 210a, emitted for instance by artificial or environmental light sources (not shown), may further interfere with the operation of (e.g., saturate) the physiological sensor 202, or otherwise reduce the quality of the measured PPG signal. Conversely, in the embodiment of FIG. 2B, the wearable device 104 is excessively tightened, resulting in the physiological sensor 202 being secured to the user's skin 106 with excessive contact force. As a result, the tissues 206 and blood vessels 208 are compressed and the blood vessels 208 constricted (e.g., leading to vasoconstriction), which may result in masking of valuable physiological information from the raw physiological sensor signal 108. In other words, while the parasitic light 210b appears less present in the embodiment of FIG. 2B, the excessive degree of compression may adversely affect blood circulation through the tissues 206 and blood vessels 208 and may therefore reduce the quality of the measured physiological sensor (i.e. PPG) signal 108.

In order to improve the quality of physiological measurements, it is therefore desirable to properly select and adjust the tightness level of the wearable device 104. As used herein, the term “optimal tightness level” (or “optimal coupling”, also referred to herein as a “target coupling”) refers to a tightness level (i.e. a coupling between the wearable device 104 and a body part of the user 102) at which the quality of the physiological sensor signal 108 is maximized (i.e. reaches a maximum value), while providing an acceptable level of comfort for the user 102. Tightness levels other than (i.e. above or below, within a pre-determined tolerance or threshold) the optimal tightness level are considered “sub-optimal”, where the term “sub-optimal tightness level” may refer to a tightness level at which the quality of the physiological sensor signal 108 is degraded (compared to the sensor signal quality at the optimal tightness level), while providing an unacceptable level of comfort (i.e. providing discomfort) for the user 102. FIGS. 2A and 2B illustrate two instances of a sub-optimal tightness level, namely a first sub-optimal tightness level, referred to herein as “too loose” (meaning that the tightness level is below the optimal tightness level by a first threshold), and a second sub-optimal tightness level, referred to herein as “too tight” (meaning that the tightness level is above the optimal tightness level by a second threshold), respectively.

Referring back to FIG. 1, the physiological sensor signal 108 generated by the physiological sensor (reference 202 in FIGS. 2A and 2B), for instance the PPG signal, may be output by the physiological sensor 202 to a signal acquisition and processing module 110 communicatively coupled with the wearable device 104 by any suitable communications (e.g., wired or wireless) means. In one embodiment, the signal acquisition and processing module 110 may be configured to pre-process the raw physiological sensor signal 108 received from the physiological sensor 202. The signal acquisition and processing module 110 may indeed be configured to down-sample the physiological sensor signal 108, attenuate artifacts and/or facilitate subsequent extraction of features (or properties) of interest from the physiological sensor signal 108. The signal acquisition and processing module 110 may then extract one or more features of interest from the physiological sensor signal 108 (optionally pre-processed). The feature(s) of interest may be extracted from the physiological sensor signal 108 over a pre-determined time window (e.g., a fixed window size of 0.5 seconds or 1 second may be used). In one embodiment, heart rate is the feature of interest that is extracted from the physiological sensor signal 108. Other embodiments may apply, depending on the application, the sensor type, and/or the sensor signal acquired.

As used herein, the term “features of interest” (or “properties of interest”) refers to any value or quantitative measure derived from a signal and that characterize the signal. At least some of the features of interest correspond to statistical parameters of the signal. A feature of interest characterizes, for instance, a data set, a probability distribution, or a spectral density function or spectrum. In some embodiments, the features of interest extracted from the physiological sensor signal 108 may include, but are not limited to, mean, median, variance, standard deviation, inter-quartile interval, skewness, kurtosis, root mean square, energy, entropy, approximate entropy, maximum slope, singular value decomposition (SVD), Sym8 wavelet transform energy at levels 1 to 9 (and more particularly at level 4), maximum, minimum, first statistical moment, second statistical moment, third statistical moment, fourth statistical moment (and more particularly the third statistical moment), median frequency, total spectral power (in a range of 0 Hz to about 10 Hz), relative spectral power (in the range of 0 Hz to about 10 Hz), peak amplitude (in the range of 0 Hz to about 10 Hz), mean of first derivative, standard deviation of first derivative, number of median crossings, power spectral density at 1, 3, 5, 7, 9, 13, 17, 21 and 29 Hz (and more particularly at 7 Hz), coefficients from third order autoregressive (AR) model, and/or number of median crossings at the instantaneous frequency of the sensor signal. It should however be understood that other features may also be considered as being of interest.

Once extracted, at least some of the features of interest are sent to the contact force estimation module 112 configured to estimate the contact force between the wearable device 104 and the user's skin 106. The contact force estimation module 112 is configured to use the received feature(s) of interest as predictor(s) for contact force estimation. In one embodiment, the contact force estimation module 112 may implement a supervised learning approach in which the predictor(s) selected among the extracted feature(s) of interest are used as part of a machine learning classification model to classify or predict the contact force. In other words, the predictor(s) are used as input to the machine learning classification model and the model's output is the estimated (or predicted) contact force between the wearable device 104 and the user's skin 106.

The machine learning classification model described herein, such as the one implemented by the contact force estimation module 112, may be trained using suitable labeled training data and a suitable optimization process to minimize a loss function. In one embodiment, the machine learning classification model implemented by the contact force estimation module 112 may be trained in advance prior to the deployment of the system 100. In other embodiments, the machine learning classification model may be trained in real-time, based on live (i.e. real-time) operational data from the user 102. Still other embodiments may apply. For instance, a hybrid approach of training the model partly in advance and partly in real-time may be used. Furthermore, the parameters of the machine learning classification model may be continuously tuned to improve the accuracy of the model, for example by enhancing the data fed as input to the machine learning classification model. Machine learning refinement may occur at different stages of the model and at different time points (e.g., using feedback to refine the machine learning classification model after deployment of the system 100).

In one embodiment, the machine learning classification model is a bagged tree model. In order to reduce computational load while maintaining predictive power, a minimal viable number of predictors may be found by sequentially training models that include an increasingly large subset of the above-mentioned features of interest. Step forward feature selection may be used at each iteration to determine the smallest viable set of predictor variables to use in the model. The performance at each iteration may be estimated using root mean squared error (RMSE) and k-fold cross-validation (CV), for instance 5-fold cross-validation. The number of predictors to include in the model (i.e. to be extracted by the signal acquisition and processing module 110) may be determined to be the number at which the inclusion of an additional feature reduces the RMSE by less than a pre-determined percentage of the current value, for instance 1%. As indicators of model performance, mean absolute errors (MAE), RMSE and the coefficient of determination r² may be used. Alternative embodiments using other machine learning classification models including, but not limited to, logistic regression, support vector machines (SVMs), decisions trees, Naïve Bayes, and Random Forest approaches, may also apply.

As illustrated in FIG. 1, a feedback module 118 may be used to provide objective tightness adjustment feedback to the user 102, the feedback optimized for physiological sensing effectiveness and provided using any suitable feedback modality. In particular, the feedback module 118 may be used to guide the user 102 to manually adjust the tightness level of the wearable device 104, in order to achieve an appropriate and consistent coupling force for a given application. For this purpose, in one embodiment, the feedback module 118 may be configured to formulate a tightness recommendation problem in which a number of classes is defined and the estimated contact force (obtained from the contact force estimation module 112) is classified into one of the classes. The feedback module 118 may be configured to dynamically update the classification in response to real-time adjustments of the wearable device 104 (e.g., manual tightness adjustments made by the user 102). The adjustment feedback may therefore be updated in real-time, as the user 102 adjusts the tightness of the wearable device 104.

In one embodiment, the tightness recommendation problem may be formulated as a two-class classification task, in which the two classes comprise an “optimal” class and a “too loose” class. In this embodiment, the “optimal” class comprises a range of contact forces that correspond to the optimal tightness level, with the signal quality of the physiological signal being maximized at the optimal tightness. The “too loose” class comprises a range of contact forces that correspond to a sub-optimal tightness level below the optimal tightness level. A first threshold between the “optimal” class and the “too loose” class may be introduced as a transition from the “optimal” to the “too loose” tightness levels. During use of the wearable device 104, oscillations between the at least two classes, due to noise or relaxation of the strap 120 of the wearable device 104, could negatively impact the user's experience. As such, hysteresis may be introduced, for instance by including a second threshold lower than the first threshold for transitions from “optimal” to “too loose”. The first and second thresholds may be determined in any suitable manner. In some embodiments, a third “too tight” class may be used, the “too tight” class comprising a range of contact forces that are deemed to correspond to a sub-optimal tightness level greater than the optimal tightness level. Still, it should be understood that the number of classes may vary depending on the application, such that other classes may apply.

With continued reference to FIG. 1, in some embodiments, different feedback modalities 114 may be employed by the feedback module 118 to communicate the tightness level (or coupling rate) of the wearable device 104. In some embodiments, a short text prompt may be generated by the feedback module 118 and presented on a display (not shown) of the wearable device 104 to suggest tightness adjustments. It should however be understood that the text prompt 116 may be output on any other suitable output device, such as on a display of the user's smartphone (not shown), or the like. For example, the text prompt 116 may comprise the following: “For better heart rate measurements, try tightening your watch strap!” or “The band is tight enough!”. In other embodiments, the system 100 may detect that the wearable device 104 does not uniformly contact the user's body part (e.g., is not equally pressed down on all sides). For example, the wearable device 104 may be a large smartwatch that is angled when coupled to a small wrist. This may be detected using the contact force estimated based on a pressure differential measured using a first and a second physiological sensor 202 respectively positioned on sides (e.g., top and bottom) sides of the body part. The lack of uniform contact may also or additionally be detected using the contact force estimated based on one or more additional sensor signals obtained from one or more additional sensors configured to measure the angular direction of the wearable device 104 and/or body part orientation. The feedback module 118 may then generate specific adjustment feedback to cause the user 102 to properly adjust the wearable device 104. For example, the feedback module 118 may cause the following text prompt 116 to be output: “Make sure the bottom of the watch is pressed down equally as much as the top!”. Any other suitable text prompt 116 may apply, depending on the contact force value estimated by the contact force estimation module 118.

In other embodiments, the tightening adjustment feedback may be provided by the feedback module 118 using vibrations. For example, when the user 102 is securing the wearable device 104 to his or her body, vibrations may be initially output having a given vibration parameter associated therewith. The vibration parameter may be initially set at a given value in order to encourage the user 102 to loosen the wearable device 104 (or conversely to tighten the wearable device 104). The vibration parameter may be modified over time (as a result of manual adjustment of the wearable device's tightness), as the user 102 approaches optimal tightness. In other words, the vibration parameter may be indicative of a current (or actual) coupling between the wearable device 104 and the user's body part and the coupling may impact the vibration parameter.

The vibration parameter may comprise any suitable parameter that characterizes the vibrations including, but not limited to, vibration intensity, a number of pulses of the vibrations, a tempo (or duration) of the pulses, a vibration frequency, or any combinations thereof. In one embodiment, the vibration parameter may be set at a first value when the actual coupling is above the optimal coupling (i.e. the coupling is too tight), and at a second value when the actual coupling is below the optimal coupling (i.e. the coupling is too loose). For example, a first number of pulses (e.g., five (5) pulses) may be output to indicate that the wearable device 104 is currently coupled to the user's body part too tight while a second number of pulses (e.g., two (2) pulses) lower than the first number of pulses may be output to indicate that the current coupling between the wearable device 104 and the user's body part is too loose. In another example, the tempo of the vibrations may be varied such that several long pulses may be output for a coupling that is too tight and several short pulses for a coupling that is too loose, where long pulses refers to pulses having a duration above a predetermined threshold and short pulses refers to pulses having a duration below a predetermined threshold. In yet another example, the frequency of the vibrations may be varied such that vibrations at a first frequency (e.g., 160 Hz) may be output to indicate a coupling that is too tight and vibrations at a second frequency (e.g., 80 Hz) lower than the first frequency may be output for a coupling that is too loose.

Vibrations may also be output at a high intensity (i.e. an intensity above a pre-determined threshold) to indicate that the wearable device 104 is currently coupled to the user's body part too tight (or conversely at a low intensity, i.e. an intensity below the threshold, to indicate that the wearable device 104 is currently coupled to the user's body part too loose) and gradually decrease (or increase) over time as the user 102 approaches optimal tightness. Other embodiments may apply. In addition, it should be understood that a combination of text prompts and vibrations may used. It should also be understood that the feedback module 118 may provide the adjustment feedback in any other suitable manner including, but not limited to, using graphics (e.g., a graphical prompt), sound (e.g. an audio prompt or the like), and light.

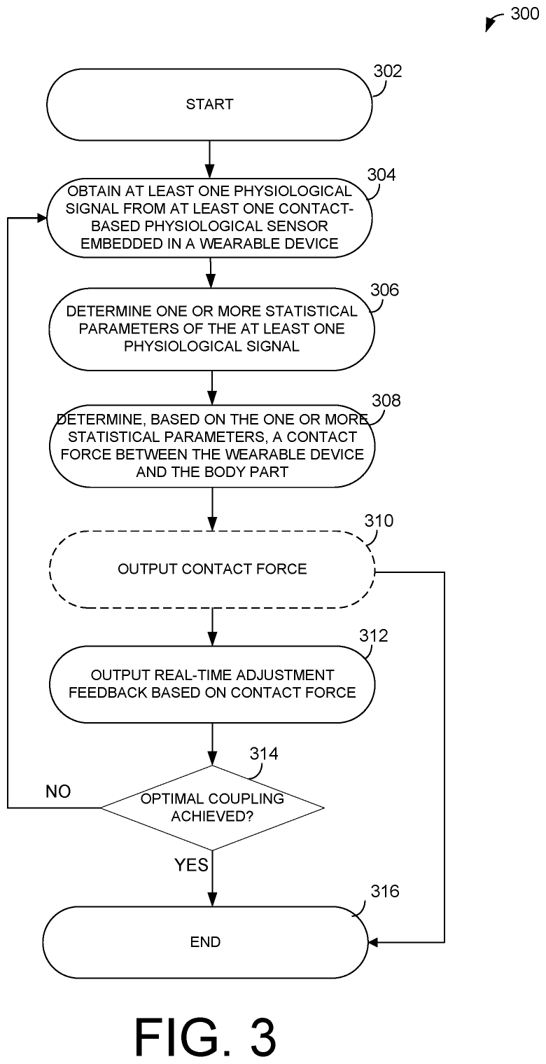

Referring now to FIG. 3 in addition to FIG. 1, a method 300 for providing contact force estimation and adjustment feedback for a wearable device, such as the wearable device 104 of FIG. 1, secured to a body part of a user, such as the user 102, will now be described. After start (step 302), at least one physiological signal (indicative of a measurement of at least one physiological parameter associated with the user) is obtained from at least one contact-based physiological sensor embedded in the wearable device (step 304). At step 306, one or more statistical parameters (i.e. properties of interest) of the at least one physiological signal are obtained, in the manner described herein above with reference to FIGS. 1, 2A, and 2B. At step 308, a contact force between the wearable device and the user's body part is determined based on the one or more statistical parameters determined at step 306. The contact force may be estimated using a machine learning classification model, in the manner described herein above. The contact force may then be output at step 310. At step 312, real-time adjustment feedback is output based on the contact force, using any suitable feedback modality as described herein above. The next step 314 may then comprise assessing whether an optimal coupling (or tightness level) between the wearable device and the user's body part has been achieved. If this is the case, the method 300 may end (Step 316). Otherwise, the method 300 may repeat (e.g., proceed back to step 302 where at least one physiological signal is obtained), subsequent to tightness adjustments being made (e.g., manual tightness adjustments performed by the user, for instance by loosening or tightening the wearable device).

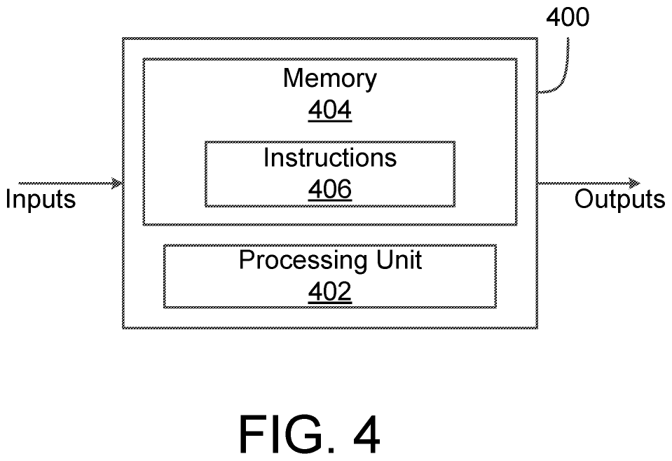

FIG. 4 illustrates an example computing device 400, which may be used to implement the system 100 of FIG. 1 and/or the method 300 of FIG. 3. The computing device 400 comprises a processing unit 402 and a memory 404 which has stored therein computer-executable instructions 406. The processing unit 402 may comprise any suitable devices configured to implement the functionality of the system 100 and/or the method 300 such that instructions 406, when executed by the computing device 400 or other programmable apparatus, may cause the functions/acts/steps performed by the system 100 and/or the method 300 as described herein to be executed. The processing unit 402 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, custom-designed analog and/or digital circuits, or any combination thereof.

The memory 404 may comprise any suitable known or other machine-readable storage medium. The memory 404 may comprise non-transitory computer readable storage medium, for example, but not limited to, an electronic, magnetic, optical, electromagnetic, infrared, or semiconductor system, apparatus, or device, or any suitable combination of the foregoing. The memory 404 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 404 may comprise any storage means (e.g., devices) suitable for retrievably storing machine-readable instructions 406 executable by processing unit 402.

In one embodiment, the systems and methods described herein may be used in medical applications, to achieve optimal heart rate measurement quality. This may result in greater confidence placed in the collected signals. Due to the increase in signal quality, psycho-physiologically attentive systems may be able to better capture the internal states of their users and adapt their behavior appropriately.

In other embodiments, the systems and methods described herein may be used in haptic applications. The consistent perception of haptic effects is indeed dependent on achieving robust mechanical coupling between the haptic system and a user's skin. Changes in coupling may cause failure to perceive and misinterpretation of haptic cues. The systems and methods described herein may be employed to adjust properties of a stimulus presented by a haptic system, to ensure consistent perception of the stimulus by users. Knowledge that a wearable device is worn at a consistent tightness may allow a more informed exploration of in-the-wild tactile effect perception.

In other embodiments, the systems and methods described herein may be applied to next generation head-mounted displays (HMD), which may be equipped with physiological and behavioral sensors (e.g., gaze and motion tracking sensors). The systems and methods described herein may be employed to ensure robust coupling between the HMD and the user's head, facilitating the collection of high quality physiological signals and ensuring that the device is appropriately positioned to maintain optical focus.

In yet other embodiments, the systems and methods described herein may be employed to provide pressure-based continuous user input on wearable and video game controllers. For example, a smartwatch typically only allows discrete touch input (i.e. touching or not, position of the touch). Using the systems and methods described herein, smartwatches may integrate an additional input dimension (i.e. pressure). This may be useful to enter continuous information without having to interact with small sliders, and the like. This may also allow users to provide inputs through clothing (e.g., gloves) that would typically preclude smartwatch interactions. The systems and methods described herein may also be used as a new input source in handheld video game controllers, to provide a continuous input source for games.

The systems and methods described herein may also prove useful in smart garment applications. The fit of smart clothing equipped with physiological sensors is generally estimated using traditional sizing charts. The systems and methods described herein may provide an objective source of information to determine how a smart garment fits a specific user's body, thereby offering benefits to the quality of physiological signals estimation. If measurements are performed at various locations on the body, the systems and methods described herein may be used to modify haptic or other stimulation at or near these different locations on the body to provide a consistent haptic effect across users.

For activity detection applications, the systems and methods described herein may provide a complementary source of information (e.g., to inertial measurement units or IMUs) to improve accuracy in activity recognition applications and in within-exercise activity count (e.g., counting the number of repetitions, recognizing specific activities such as tennis serves versus backhands, and the like).

In other embodiments, the systems and methods described herein may be implemented in self-tightening systems. For example, a wristband, belt or shoe may automatically tie or tighten itself using actuators. The systems and methods described herein may be used as an input to a system controlling the automated tightness adjustment process.

The above description is meant to be exemplary only, and one skilled in the art will recognize that changes may be made to the embodiments described without departing from the scope of the present disclosure. Still other modifications which fall within the scope of the present disclosure will be apparent to those skilled in the art, in light of a review of this disclosure.

Various aspects of the systems and methods described herein may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Although particular embodiments have been shown and described, it will be apparent to those skilled in the art that changes and modifications may be made without departing from this invention in its broader aspects. The scope of the following claims should not be limited by the embodiments set forth in the examples, but should be given the broadest reasonable interpretation consistent with the description as a whole.

What is claimed is:

1. A contact force determination method, comprising:
obtaining, at a computing device, at least one physiological signal indicative of at least one physiological parameter of a user, the at least one physiological signal obtained from at least one contact-based physiological sensor embedded in a wearable device secured to a body part of the user;
determining, at the computing device, one or more statistical parameters of the at least one physiological signal;
determining at the computing device, based on the one or more statistical parameters, a contact force between the wearable device and the body part; and
outputting, at the computing device, the contact force as determined.

2. The contact force determination method of claim 1, wherein obtaining the at least one physiological signal comprises obtaining a photoplethysmogram (PPG) signal from an optical heart rate monitor.

3. The contact force determination method of claim 2, further comprising obtaining at least one additional sensor signal from at least one additional sensor embedded in the wearable device, and determining the contact force based on the at least one additional sensor signal.

4. The contact force determination method of claim 3, wherein the at least one additional sensor comprises a skin conductance sensor, a motion sensor, an accelerometer, a gyroscope, and/or an altimeter.

5. The contact force determination method of claim 1, wherein determining the contact force comprises selecting at least some of the one or more statistical parameters and using the at least some of the one or more statistical parameters as predictors in a machine learning classification model trained to estimate the contact force based on the predictors.

6. The contact force determination method of claim 1, further comprising outputting real-time adjustment feedback based on the contact force as determined, the adjustment feedback comprising one or more indications to achieve a target coupling between the wearable device and the body part at which a quality of the at least one physiological signal is maximized.

7. The contact force determination method of claim 6, wherein outputting the adjustment feedback comprises outputting at least one text prompt, at least one audio prompt, and/or at least one graphical prompt indicative of at least one tightness adjustment for the wearable device, the at least one tightness adjustment to be performed to achieve the target coupling.

8. The contact force determination method of claim 6, wherein outputting the adjustment feedback comprises causing one or more vibrations of the wearable device, the one or more vibrations having associated therewith at least one vibration parameter indicative of an actual coupling between the wearable device and the body part relative to the target coupling.

9. The contact force determination method of claim 8, wherein the at least one vibration parameter is set at a first value when the actual coupling is above the target coupling by a first threshold, and at a second value when the actual coupling is below the target coupling by a second threshold, the first value greater than the second value.

10. The contact force determination method of claim 9, wherein the actual coupling being above the target coupling corresponds to a tight coupling between the wearable device and the body part and the actual coupling being below the target coupling corresponds to a loose coupling between the wearable device and the body part.

11. The contact force determination method of claim 8, wherein the at least one vibration parameter comprises an intensity of the one or more vibrations, a number of pulses of the one or more vibrations, a tempo of the one or more vibrations, and/or a frequency of the one or more vibrations.

12. A contact force determination system comprising:
a processing unit; and
a non-transitory computer-readable medium having stored thereon program instructions executable by the processing unit for:
obtaining at least one physiological signal indicative of at least one physiological parameter of a user, the at least one physiological signal obtained from at least one contact-based physiological sensor embedded in a wearable device secured to a body part of the user;
determining one or more statistical parameters of the at least one physiological signal;
determining, based on the one or more statistical parameters, a contact force between the wearable device and the body part; and
outputting the contact force as determined.

13. The contact force determination system of claim 12, wherein the instructions are executable by the processing unit for obtaining the at least one physiological signal comprising obtaining a photoplethysmogram (PPG) signal from an optical heart rate monitor.

14. The contact force determination system of claim 13, wherein the instructions are executable by the processing unit for obtaining at least one additional sensor signal from at least one additional sensor embedded in the wearable device, and determining the contact force based on the at least one additional sensor signal.

15. The contact force determination system of claim 14, wherein the at least one additional sensor comprises a skin conductance sensor, a motion sensor, an accelerometer, a gyroscope, and/or an altimeter.

16. The contact force determination system of claim 12, wherein the instructions are executable by the processing unit for determining the contact force comprising selecting at least some of the one or more statistical parameters and using the at least some of the one or more statistical parameters as predictors in a machine learning classification model trained to estimate the contact force based on the predictors.

17. The contact force determination system of claim 12, wherein the instructions are executable by the processing unit for outputting real-time adjustment feedback based on the contact force as determined, the adjustment feedback comprising one or more indications to achieve a target coupling between the wearable device and the body part at which a quality of the at least one physiological signal is maximized.

18. The contact force determination system of claim 17, wherein the instructions are executable by the processing unit for outputting the adjustment feedback comprising outputting at least one text prompt, at least one audio prompt, and/or at least one graphical prompt indicative of at least one tightness adjustment for the wearable device, the at least one tightness adjustment to be performed to achieve the target coupling.

19. The contact force determination system of claim 17, wherein the instructions are executable by the processing unit for outputting the adjustment feedback comprising causing one or more vibrations of the wearable device, the one or more vibrations having associated therewith at least one vibration parameter indicative of an actual coupling between the wearable device and the body part relative to the target coupling.

20. A non-transitory computer-readable medium having stored thereon program instructions executable by a processor for:
obtaining at least one physiological signal indicative of at least one physiological parameter of a user, the at least one physiological signal obtained from at least one contact-based physiological sensor embedded in a wearable device secured to a body part of the user;
determining one or more statistical parameters of the at least one physiological signal;
determining, based on the one or more statistical parameters, a contact force between the wearable device and the body part; and
outputting the contact force as determined.
